(19) United States
(12) Patent Application Publication
(10) Pub. No.: US 2014/0349363 A1
(43) Pub. Date: Nov. 27, 2014

(54) Biogas Plant And Method For Operating A Biogas Plant

(71) Applicant: KOMPOFERM GmbH, Marienfeld (DE)

(72) Inventor: Karlgünter Eggersmann, Marienfeld (DE)

(21) Appl. No.: 14/348,180

(22) PCT Filed: Sep. 29, 2011
(86) PCT No.: PCT/EP2011/004853

§ 371 Date: Jun. 23, 2014

(51) Int. Cl. C12M 1/107; C12P 5/02

(52) U.S. Cl. CPC C12M 21/04; C12P 5/023
USPC 435/167; 435/286.6

(57) ABSTRACT

A biogas plant has at least one fermenter (1) and a first gas store (3) and a second gas store (5) for storing biogas. The gas store (3) is connectable to a first biogas conduit (4) that is connectable to a gas outlet (7) of at least one fermenter (1) while the second gas store (5) is connectable to a second biogas conduit (6) that is connectable to a gas outlet (7) of the at least one fermenter (1). A biogas utilization appliance (2) is connectable to the first biogas store (3) and/or to the first biogas conduit (4) and to the second biogas store (5) and/or to the second biogas conduit (6). The biogas plant has a control appliance (8) for controlling the quantitative ratios of the biogas streams conducted from the biogas conduits (4, 6) and/or the biogas stores (3, 5) to the biogas utilization appliance (2).

### TECHNICAL FIELD

The invention relates to a biogas plant with at least one fermenter for producing biogas and a method for operating a biogas plant.

### BACKGROUND OF THE INVENTION

Biogas plants of this type are used to generate biogas containing methane, for example using a block heat and power plant. In this case the fermenters operate in batch mode, i.e., continuous operation of such a fermenter between the filling and unloading of the fermenter with the biomass to be fermented or that has already been fermented does not take place. Instead, at the beginning and end of the biomass fermentation, startup and shutdown phases occur, during which gases or gas-air mixtures are produced which cannot be utilized efficiently, primarily because of their inadequate methane content. At this time these gas mixtures are either flared off or subjected to a different type of waste air treatment. As a result of the losses of potentially usable methane, the efficiency of such a plant is reduced. Additional efficiency-reducing losses in conventional plants arise in that the temperature in the fermenter is higher than the temperature at which the biogas must be supplied to conventional utilization equipment. Consequently heat losses in the fermenter occur, and on the other hand, it is necessary to use coolers for the biogas.

### SUMMARY OF THE INVENTION

The invention is now based on the task of disclosing a biogas plant and a method for operating a biogas plant which avoid or at least reduce the losses described and thus make higher efficiency of the biogas plant possible.

According to the invention it is provided that biogas obtained in a fermenter, having a methane content below a first preselectable limit value, is stored temporarily in the first gas store. This is the biogas that forms during the startup and shutdown phases in conventional plants and generally must be disposed of. This biogas with methane content below the first preselectable limit value will also be called “weak gas” in the following. Advantageously this weak gas will be mixed with biogas that has a methane content above a preselectable limit value, called “good gas” in the following, and will operate a biogas utilization appliance with a mixture of good gas and weak gas, called “mixed gas” in the following.

To permit implementation of the method, the biogas plant according to the invention has at least two gas stores, so that a first gas store is available for storing weak gas and a second gas store is available for storing good gas. The gas stores can be connected over biogas conduits to gas outlets of the at least one fermenter, and the biogas utilization appliance is connected to the biogas store and/or the biogas conduit in such a manner that it is possible on one hand to take biogas from the first gas store or the conduits supplying it, and on the other hand to take biogas from the second gas store or the conduits supplying this, and feed the two gases to the biogas utilization appliance simultaneously.

According to the invention the biogas plant has a control appliance for controlling the mixing ratios of the biogas streams conducted from the biogas conduits and/or the biogas stores to the biogas utilization appliance. By means of this interconnection it is possible to feed usable gas mixtures to the biogas utilization appliance, even if gas is temporarily produced during the startup or shutdown phases of the fermenter or one of the fermenters with a methane content that would normally be too low for use.

By means of the gas store it is thus possible to store good gas or weak gas temporarily, so that on average, usable biogas can always be generated as a mixed gas over the operating period of the biogas plant, even from a weak gas fraction that is temporarily too high and is then stored in the weak gas store in the interim. The weak gas stored in the interim can then be taken from the store during the operating phases with high good gas fraction and added to the mixed gas. In the opposite case it is likewise possible, during phases with a high good gas fraction, to store this temporarily and remove it from the reservoir in phases with insufficient good gas fraction to obtain a usable mixed gas.

Advantageously in this process the quantitative ratios of good gas and weak gas are regulated such that the methane content of the mixed gas ranges above a third preselectable limit value. Preferably the regulation is accomplished in that it takes place depending on the methane content of good and/or weak gas, so that in the case of fluctuating methane contents in good gas and weak gas, it is nevertheless assured that the methane content of the mixed gas exceeds the third preselectable limit value.

Preferably, therefore, the biogas plant has a measuring device for measuring the methane content which can be connected to the biogas conduits and/or the biogas stores, so that advantageously values above the methane contents of both the gas currently being produced in the fermenters and the gases stored in the biogas stores and/or of the mixed gas supplied to the biogas utilization appliance are available.

Advantageously the first limit value is selected such that the methane content in the first biogas store, thus the methane content of the weak gas, is below the lower explosion limit, which depends on the oxygen content. In this way the capital costs can be reduced as a result of the lower requirements for explosion prevention of the plant.

Advantageously the second limit value, thus the lower limit value for the methane content of the good gas, is 37%, preferably 52%. It is also advantageous if the third limit value, thus the limit value for the minimum methane content of the mixed gas, is 35%, preferably 50%. Methane contents above this limit value present possibilities for utilization of established types of block heat and power plants and permit operation of the plant largely without interruption.

Advantageously biogas or weak gas can be conveyed from a fermenter back into it and thus kept in circulation at least in phases or cyclically. For this purpose the fermenter has a flushing gas inlet, preferably in the area of the bottom of the fermenter, which can be connected to the corresponding biogas conduit and thus makes it possible to flush the biomass with good gas and/or weak gas to loosen it up and thus guarantee better drainage in the biomass.

Preferably the flushing gas inlet can also be connected to a fresh air feed conduit, which permits fresh air flushing of the fermenter, especially during the startup or shutdown operation. It is further advantageous if the flushing gas inlet can also be connected to the weak gas and/or good gas store to also carry out the corresponding flushing treatments with gas from the reservoir, for example if the gas, based on the requirements imposed on the gas mixture supplied for biogas utilization, cannot be taken directly from a fermenter without interfering with the operation of the biogas utilization appliance.

It is particularly advantageous if, during the startup or shutdown of a fermenter, weak gas is supplied to the fermenter through the flushing gas inlet, since it then becomes possible to obtain and utilize good gas in this fermenter during these phases.

Advantageously the biogas plant has an outlet air/outlet gas conduit which can be connected with the gas outlet of the at least one fermenter and leads to an outlet air treatment plant. This makes it possible to specify limit values independently of one another in such a way that gas mixtures that form in fermenters and based on their composition cannot be assigned either to the good gas or the weak gas can be safely disposed of.

Advantageously at least one of the gas stores is arranged above the at least one fermenter, so that it is adjacent to it over a large area. Preferably at least 50% of the surface of the at least one fermenter is covered in this case. Particularly advantageously the entire surface of the at least one fermenter or especially advantageously, of all fermenters present are covered by the gas store(s). As a result of this design, particularly advantageous thermal insulation of the fermenter can be achieved throughout most of the year, wherein at the same time, as a result of the flat design of the gas store(s), these have a large interface with the environment, which over the annual average permits good gas cooling without requiring a footprint exceeding the set-up space of the fermenter.

### BRIEF DESCRIPTION OF THE DRAWINGS

In the following drawings, the invention will be explained schematically in further detail based on FIGS. 1 to 10 wherein:

FIG. 1 is a schematic process flow diagram of an example of a biogas plant according to the invention during the production of good gas in two fermenters with simultaneous storage of weak gas from a third fermenter;

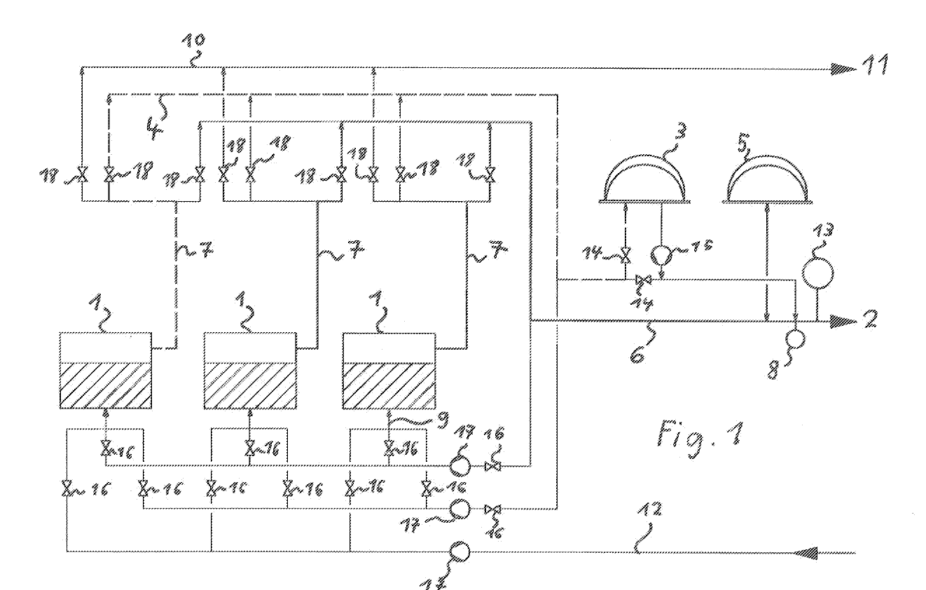

FIG. 2 is a flow diagram of the plant from FIG. 1 wherein weak gas is being mixed with the good gas before it is used;

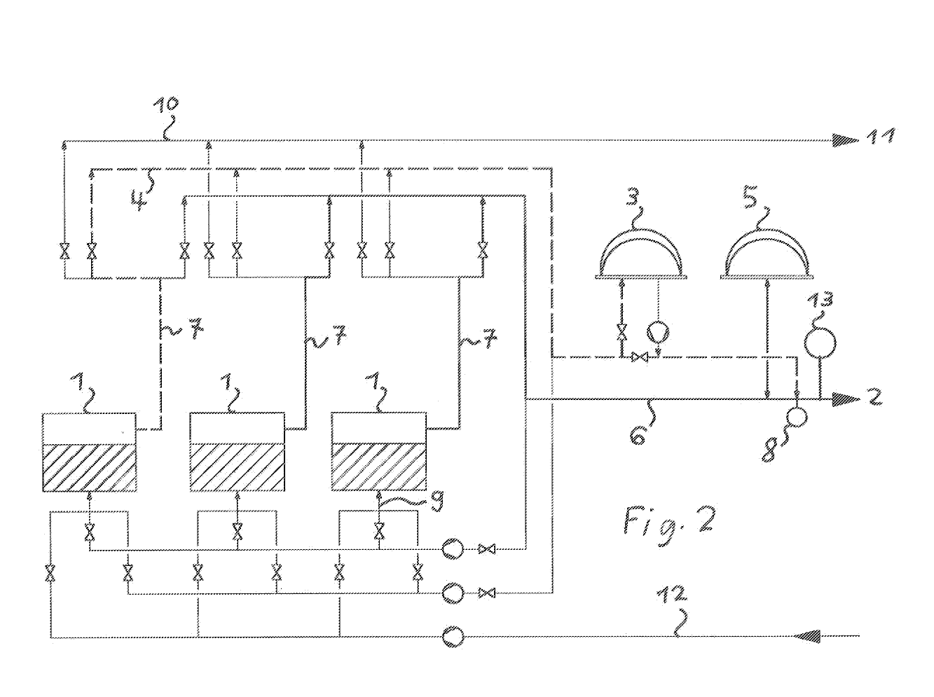

FIG. 3 is a flow diagram of the plant from FIGS. 1 and 2 during the introduction of weak gas from the weak gas store into a fermenter with simultaneous removal of good gas from all fermenters;

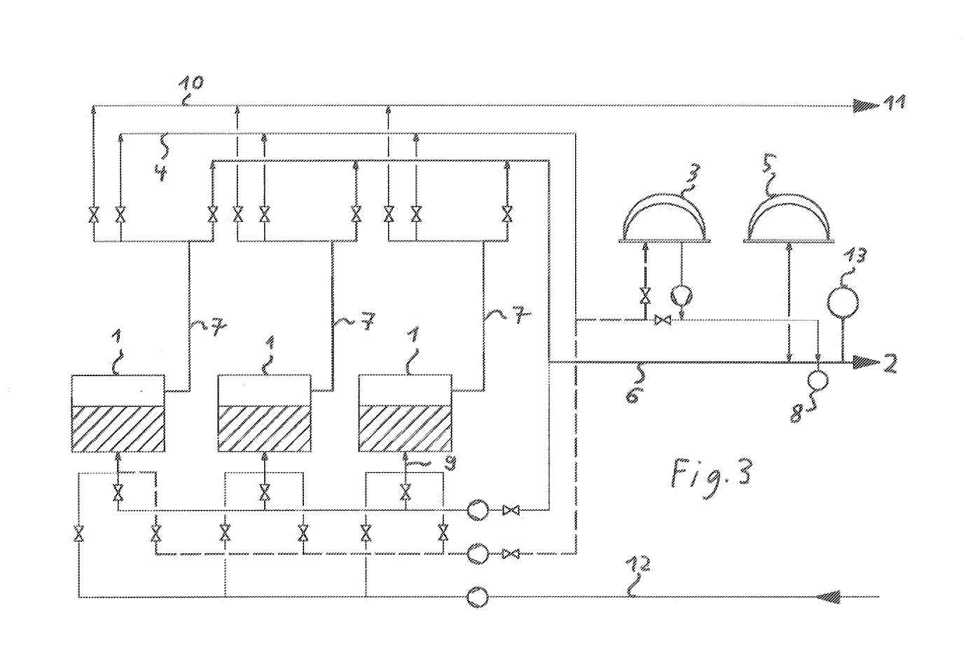

FIG. 4 is a flow diagram of the plant shown in FIGS. 1 to 3 during the production and utilization of gas in two fermenters with simultaneous storage of weak gas from one fermenter, wherein part of the good gas stream is returned to the fermenter producing good gas;

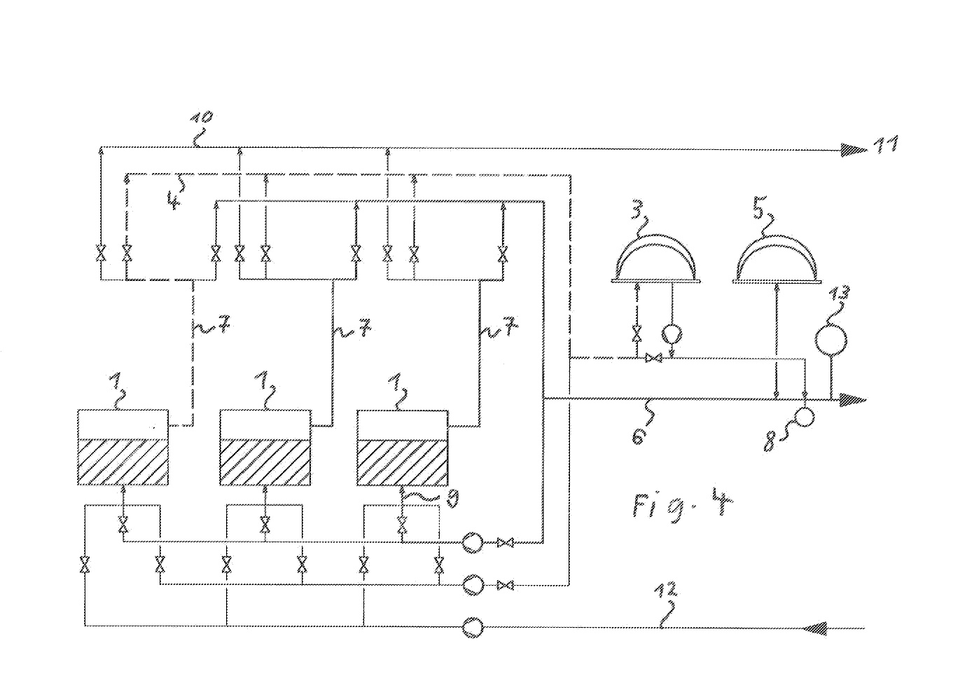

FIG. 5 is a flow diagram of the plant shown in FIGS. 1 to 4 during the production of good gas in two fermenters and the production of weak gas in one fermenter, wherein good gas and weak gas are utilized as a mixed gas and the weak gas fraction in the mixed gas is regulated by removal and/or addition of the weak gas to or from the weak gas store;

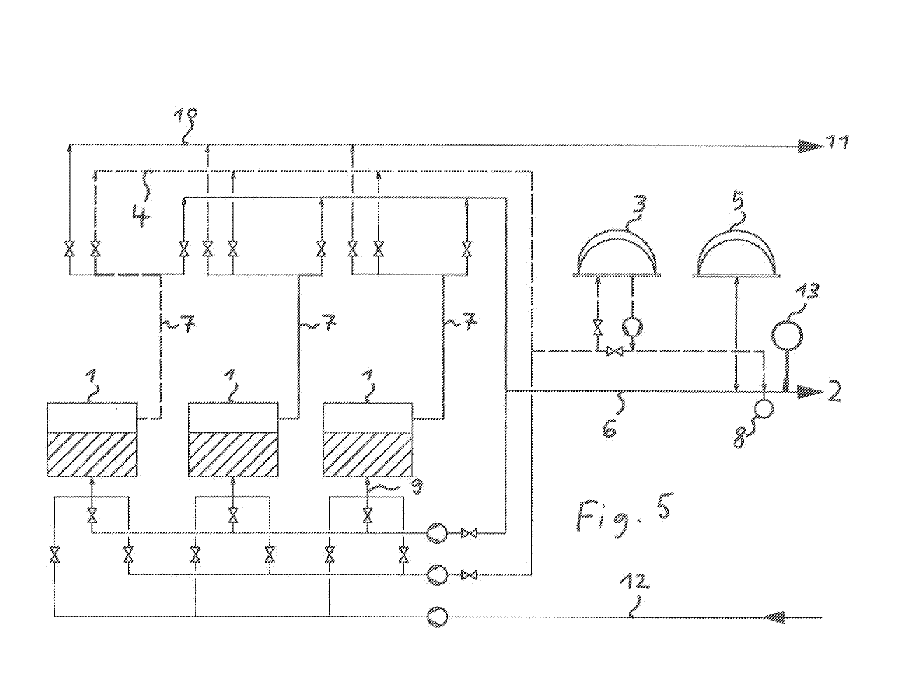

FIG. 6—a flow diagram of the plant from FIGS. 1 to 5, wherein one fermenter produces weak gas, which is utilized together with the good gas from another fermenter, wherein the fraction of the weak gas is regulated by means of the weak gas store, wherein part of the good gas is returned to the good gas-producing fermenter and a third fermenter is simultaneously being flushed with fresh air which is carried away through an outlet air/outlet gas conduit; and

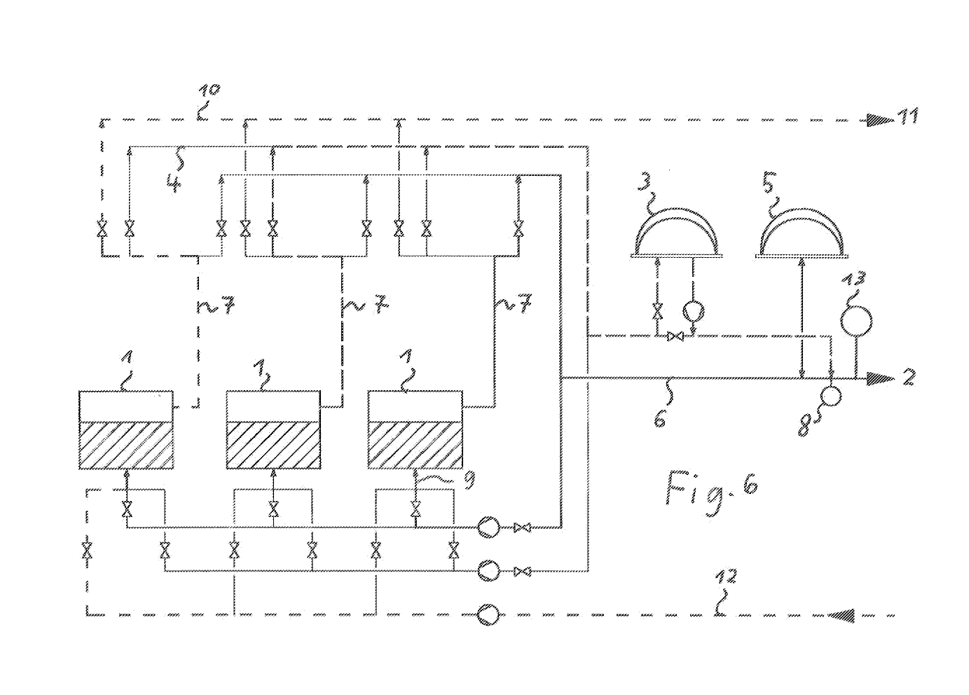

FIGS. 7-10 show different examples of setup possibilities with the placement of the gas stores on the roofs of the fermenters or alongside the fermenters.

### DETAILED DESCRIPTION OF THE PREFERRED EMBODIMENTS

The plant serving as an example of the invention has three independently operating fermenters 1, which can be connected by their gas outlet conduits 7 selectively with the first biogas conduit 4, the second biogas conduit 6 or an outlet air/outlet gas conduit 10, wherein the latter leads to an outlet air/outlet gas treatment system 11 (not shown but well known in the art). The biogas streams carried in the biogas conduits 4 and 6 can be mixed together or conveyed individually to a biogas utilization appliance 2. The example of the plant according to the invention has a control appliance 8 such as a mixing valve for mixing the two biogas streams. Furthermore a measuring device 13 which can measure the methane content of the biogas conveyed to the appliance 2 is provided in the biogas stream.

The biogas conduit 4 is, in one mode, connected to the biogas store 3, designed as a weak gas store. The biogas can be directed to the first biogas store 3 or drawn from the first biogas store 3 by way of selectively operating blocking valves 14 and a feeder device 15 which is preferably implemented as a pump. Alternatively it is possible to bypass this store by appropriately selectively operating blocking valves 14.

The second biogas conduit 6 is connected to a second biogas store 5, which is designed as an equalizing container (reservoir) for this biogas conduit in order to store some biogas to compensate for the increase and/or decrease in the biogas production and/or biogas consumption by the biogas utilization device 2.

The fermenters 1 also have gas inlets 9, which optionally and independently of one another can be connected utilizing independently operable shutoff valves 16 to the biogas conduit 4, the second biogas conduit 6 or a fresh air feed conduit 12. Conveying equipment 17 is provided in the form of one or more pumps, serving to supply an adequate pressure to the flushing gas utilized in the flushing of the fermenter 1 over the gas inlet 9, located in the area of the base of the respective fermenter. Additional shutoff valves 18 serve to connect the gas outlets 7 of the fermenters 1 selectively and independently of one another to the outlet air/outlet gas conduit 10 of the first biogas conduit 4 and/or the second biogas conduit 6.

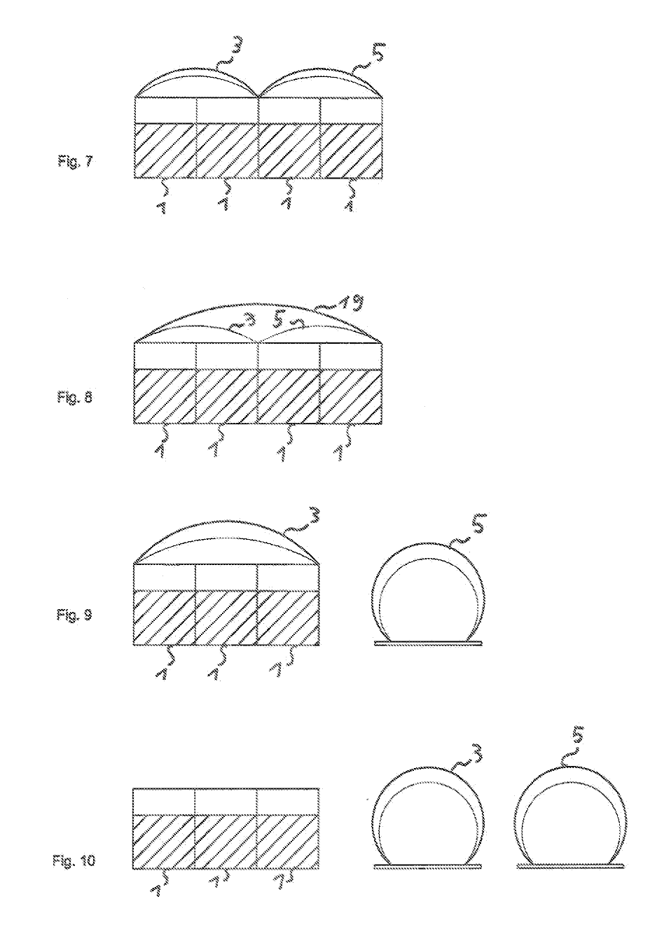

The biogas stores 3 and 5 can be set up in the conventional manner independent of the fermenters 1. However, it is also possible and particularly advantageous to operate the biogas stores such that either one of the two biogas stores, i.e., either the first biogas store 3 or the second biogas store 5, covers the roof area of the fermenter. It is likewise possible to arrange both biogas stores on a common roof area formed on one of the available fermenters 1. As shown in FIG. 7, independent biogas stores can be arranged on the roof independently of one another. However, it is also possible, as shown in FIG. 8, to provide the two biogas stores 3 and 5 with a common protective covering 19.

Modifications and substitutions by one of ordinary skill in the art are considered to be within the scope of the present invention, which is not to be limited except by the following claims.

1. Biogas plant having at least one fermenter (1) for producing biogas to be provided to a biogas utilization appliance (2), said biogas plant comprising:
a first biogas store (3) for storing biogas, said first biogas store (3) configured for being connected via a first biogas conduit (4) to a biogas outlet (7) of the at least one fermenter (1) by means of a first selectively operable valve (18), said first biogas store (3) having a biogas store outlet;
a second biogas store (5) for storing biogas, said second biogas store (5) configured for being connected via a second biogas conduit (6) to a biogas outlet (7) of the at least one fermenter (1) by means of a second selectively operable valve (18), said second biogas store (5) having a biogas store outlet;
a control appliance (8), coupled to said first biogas conduit (4), said second biogas conduit (6) and said biogas store outlets of both said first (3) and second (5) biogas stores, for controlling the quantitative ratio of biogas streams exiting from the biogas conduits (4, 6) and/or the outlets of the biogas stores (3, 5) for use by a biogas utilization appliance.

2. The biogas plant according to claim 1, wherein the at least one fermenter (1) has a flushing gas inlet (9) that can be selectively fluidly connected to a fresh air supply line (12), preferably in the area of the base of the at least one fermenter (1) by selectively operating one or more valves (16,17).

3. The biogas plant according to claim 1, wherein the at least one fermenter (1) has a flushing gas inlet (9) that can be selectively fluidly connected to the first biogas store (3) and/or the first biogas conduit (4), preferably in the area of the base of the at least one fermenter by selectively operating one or more valves (16, 17).

4. The biogas plant according to claim 1, wherein the at least one fermenter (1) has a flushing gas inlet (9) that can be selectively fluidly connected to the second biogas store (5) and/or the second biogas conduit (6), preferably in the area of the base of the at least one fermenter (1) by selectively operating one or more valves (16, 17).

5. The biogas plant according to claim 1, wherein the biogas plant includes a measuring device (13) fluidly coupled to the biogas conduits (4, 6) and/or the biogas stores (3, 5) for controlling the quantitative ratio of biogas streams exiting from the biogas conduits (4, 6) and/or the outlets of the biogas stores (3, 5) for use by a coupled biogas utilization appliance.

6. The biogas plant according to claim 1, wherein said at least one fermenter (1) has an upper exposed surface, and wherein at least one of the gas stores (3, 5) is arranged over an area of between 50% to 100% of said upper surface of the at least one fermenter (1), and preferably 100% of said upper surface of each of the at least one fermenter (1).

7. The biogas plant according to claim 1, wherein the biogas plant includes an air/outlet gas conduit (10) configured to be selectively fluidly connected with the biogas outlet (7) of the at least one fermenter (1), wherein said air/outlet gas conduit (10) is configured to be fluidly coupled to an outlet air/outlet gas treatment plant (11).

8. A method for operating the biogas plant of claim 1, wherein biogas obtained in said at least one fermenter (1), which has a methane content below a first preselectable limit value, is stored at least temporarily in a first gas store (3).

9. The method according to claim 8, wherein the biogas that has a methane content below said first preselectable limit value from said first biogas store (3) is mixed with a biogas stream obtained from said at least one fermenters (1), and wherein said mixed biogas is provided to a biogas utilization appliance (2) that is configured to be operated with said mixture of the two biogases.

10. The method according to claim 8, wherein a biogas that has a methane content above a second, preselectable limit value is stored at least temporarily in a second gas store (5).

11. The method according to claim 10, wherein a quantitative ratio of the biogases which has a methane content below said first preselectable limit value and said bio as that has a methane content above said second, preselectable limit value are regulated, preferably in such a manner that the methane content of the mixture of the two biogases ranges above a third preselectable limit value.

12. The method according to claim 8, wherein during a startup or shutdown phase of a fermenter (1), biogas that has a methane content below the first preselectable limit value is returned to this same fermenter (1), wherein simultaneously biogas that has a methane content above the second preselectable limit value is removed from this same fermenter (1).

13. The method according to claim 10, wherein biogas that has a methane content above the second preselectable limit value and/or biogas that has a methane content below the first preselectable limit value is removed from a given fermenter (1), and wherein biogas that has a methane content above the second preselectable limit value is returned into said at least one fermenter, and wherein biogas having a methane content above the second preselectable limit value and/or biogas that has a methane content below the first preselectable limit value is removed from said at least one fermenter.

14. (canceled)

15. The method according to claim 10, wherein the second, preselectable limit value is a methane content of between 37% to 52%, preferably a methane content of 52%.

16. The method according to claim 11, wherein the third preselectable limit value is a methane content of between 35% and 50%, preferably a methane content of 50%.

17. The method of claim 8, wherein the at least one fermenter (1) has a flushing gas inlet (9) disposed in a base area of the at least one fermenter (1), said flushing gas inlet (9) configured for being selectively fluidly coupled, by selectively operating one or more valves (16, 17), to at least one of a fresh air supply line (12), the first biogas store (3), the first biogas conduit (4), second biogas store (5), and the second biogas conduit (6), for flushing the biomass with biogas to loosen it up and promote better drainage in the biomass being fermented in the at least one fermenter (1).
